(19) United States
(12) Patent Application Publication
(10) Pub. No.: US 2012/0283664 A1
(43) Pub. Date: Nov. 8, 2012

(54) Use of Polyoxyalkylene Diamine-Based Polyguanidine Derivatives for Medical Articles

(75) Inventors: Thomas Riemann, Grossalmerode (DE); André Weiss, Guxhagen (DE); Jürgen Schmitt, Kirchhain (DE); Ulrich Brethauer, Korle (DE); Martin Sippel, Melsungen (DE)

(73) Assignee: B. BRAUN MELSUNGEN AG, Melsungen (DE)

(21) Appl. No.: 13/509,017

(22) PCT Filed: Nov. 12, 2010
(86) PCT No.: PCT/EP10/67406

§ 371 Date: Jul. 20, 2012

(30) Foreign Application Priority Data
Nov. 12, 2009 (DE) 102009052725.7

(51) Int. Cl. A61M 25/00; C08G 18/83; B29C 67/24; C08G 73/02

(52) U.S. Cl. 604/264; 528/422; 525/453; 264/239

(57) ABSTRACT

The invention relates to the use of at least one polymeric guanidine derivative having a biocidal effect, which can be obtained by polycondensating a guanidine acid addition salt with an amine mixture containing at least one diamine, which is selected from the group consisting of alkylene diamine and oxyalkylene diamine, as an additive in a composition for medical articles.

The invention further relates to medical articles containing polyguanidines as an additive.

The invention relates to the use of polymeric guanidine derivatives having biocidal activity in a composition for medical articles.

In addition, the invention relates to a process for preparing a medical article.

Medical articles or objects that are inserted into a patient's body, for example, intravasal catheters, breathing tubes or stents, must have as smooth a surface as possible in order to minimize complaints of the patient and deposits on the surfaces. Such medical articles and their packaging are often prepared by methods of plastics technology, for example, compression molding, extrusion molding, deep drawing and extrusion methods, from a plastic material, wherein it is tried to achieve as smooth surfaces as possible.

In order to avoid infections, it is advantageous for the medical articles to be treated with antimicrobially active agents. High demands are placed on the biocidal treatment of the medical articles, because the articles will contact body tissues and body fluids. For example, catheters, which are inserted through the skin surface into arteries and veins, but also wound or thoracic drainage tubes, are frequent sources of infection. In particular, in patients requiring indwelling urinary catheters, there is a risk of urinary tract infections, which can lead to bacterial or chronic pyelonephritis.

In the medical field, the central venous catheters, in particular, play an increasing role in medical treatments and surgical operations. Central venous catheters are employed more and more often within the scope of intensive care medicine, but also in applications, for example, in bone marrow and organ transplantations, hemodialysis or cardiothoracic surgery.

A similar infection risk exists in all devices that connect the catheters with, for example, with infusion containers outside the body, for example, connecting pieces, T pieces, couplings, filters, conduit systems, valves, syringes and multi-way stopcocks. For the purposes of this description and the claims, all these objects are referred to as “medical articles”.

However, for the medical articles, especially the catheters, not only the high demands placed on a smooth surface, for example, for avoiding or reducing platelet aggregation and biofilm formation, and on the biocidal treatment, which are supposed to prevent the growth of microbes on the surface or even kill the microbes altogether, must be ensured, but it must also be ensured that the biocidal treatment of the medical objects does not adversely affect the material properties of the medical articles. In addition, it must be ensured that the medical articles, especially if contacted with fluid, exhibit a high biocidal effectiveness on the one hand, but are not released into the fluid on the other, in order to avoid enrichment of the biocidally active substances in the body. The release of a biocidally active substance from a medical article upon fluid contact is also referred to as “leaching”.

From EP 0 229 862, medical articles made of polyurethane on the surface of which an antimicrobial agent is applied are known. From EP 0 379 269, medical articles, especially tubing, formed from a thermoplastic polymer are known that contain chlorhexidine as an antimicrobially active agent. These articles are prepared by first providing a mixture of chlorhexidine and plastic pellets of a thermoplastic polymer, which is processed into a melt in which the chlorhexidine is uniformly distributed, the melt being extruded through a die to form the medical article. However, the use of biguanide-based biocidal agents, such as chlorhexidine or polyhexamethylene biguanide, is not always satisfactory. In particular, there is a need for improvement in view of the smoothness of the surface of the medical articles and in view of the reduction or control of the leaching effect. In addition, it is the object of the present invention to provide medical articles that have been treated with biocidally active substances and are highly effective even in view of the development of resistances of the bacterial strains towards conventional antimicrobial agents. The object of the present invention is achieved by the use of polymeric guanidine derivatives as additives to materials for medical articles.

Therefore, the present invention relates to the use of at least one polymeric guanidine derivative having biocidal activity that is obtainable by the polycondensation of a guanidine acid addition salt with a mixture of amines containing at least one diamine selected from the group consisting of alkylene diamine and oxyalkylene diamine, as an additive in a composition for tubular medical articles.

It has been found that when the mentioned polymeric guanidine derivatives are used in composition containing plastic materials, especially thermoplastic polymers, wherein said plastic materials are employed for preparing medical articles, a very high smoothness of the surfaces of the plastic material can be ensured, which even exceeds that of the plastic material without the added polymeric guanidine derivatives. In addition to the excellent biocidal activity that the polymeric guanidine derivatives provide the compositions for the medical articles with, it has also been surprisingly found that the polymeric guanidine derivatives provide the compositions with a readily controllable release property (leaching effect). The controllability ranges from no release to release rates as high as several mg/m²/h.

The polymeric guanidine derivatives used according to the invention and the preparation thereof are known to the skilled person and have been described, for example, in WO-A1-01/85676 and WO-A1-06/047800.

The polymeric guanidine derivatives used according to the invention can be in the form of both homopolymers and copolymers. It is advantageous if the guanidine acid addition salt is guanidinium chloride (or guanidine hydrochloride). However, other guanidine acid addition salts based on inorganic or organic acids are also suitable, for example, the hydroxides, hydrogensulfates and acetates. Particularly suitable and effective polymeric guanidine derivatives are in the form of their hydroxide salts. These can be obtained, for example, by anion-exchange on the corresponding chloride salts.

The polymeric guanidine derivatives having biocidal activity are obtainable by the polycondensation of a guanidine acid addition salt with a mixture of amines containing at least one diamine selected from the group consisting of alkylene diamine and oxyalkylene diamine.

According to the present invention, the term “polymeric guanidine derivative” is used for guanidine derivatives in which 2 or more repeating units are contained. Thus, the term “polymer” also includes dimers, trimers or, for example, oligomers.

Preferably, the mixture of amines includes an alkylene diamine, more preferably a compound of general formula

NH₂(CH₂)ₙNH₂

in which n represents an integer of from 2 to 10, preferably 4 or 6. Preferably employed alkylene diamines have terminal amino groups. Hexamethylene diamine (hexane 1,6-diamine) is particularly preferred. The alkylene diamine can be employed in admixture with other polyamines, for example, diamines and/or triamines, in the polycondensation reaction to form copolymers.

Preferably, the mixture of amines includes at least one oxyalkylene diamine.

Suitable oxyalkylene diamines include those oxyalkylene diamines that have terminal amino groups, in particular. A preferred oxyalkylene diamine is a compound of general formula

NH₂[(CH₂)₂O)]ₙ(CH₂)₂NH₂

in which n represents an integer from 2 to 6, preferably from 2 to 5, more preferably from 2 to 4, especially 2. Oxyethylene diamines, especially diethylene glycol diamine or triethylene glycol diamine, are preferred. Polyoxypropylene diamines, especially di- or tri propylene glycol diamine, can be more preferably employed.

Preferably, the polymeric guanidine derivative is in the form of a homopolymer. In such cases, the mixture of amines consists of the alkylene diamine or an oxyalkylene diamine.

In a further preferred embodiment, the mixture of amines consists of the alkylene diamine hexamethylene diamine(hexane 1,6-diamine). In this variant, the polymeric guanidine derivative thus consists of a homopolymer, for example, poly(hexamethylene guanidinium chloride) (PHMGC).

In another preferred embodiment, the mixture of amines consists of the oxyalkylene diamine triethylene glycol diamine. Polycondensation thereof with a guanidine acid addition salt yields, for example, the homopolymer poly[2-(2-ethoxy)ethoxyethyl)guanidinium chloride].

In another preferred embodiment, the polymeric guanidine derivatives used according to the invention are in the form of copolymers. These may be either random or block copolymers. In the case of copolymers, the mixture of amines contains at least two different amines. The mixture of amines contains a first component and at least one second component, wherein
the first component is a diamine selected from the group consisting of alkylene diamine and oxyalkylene diamine, and wherein
the second component is a diamine selected from the group consisting of alkylene diamine and oxyalkylene diamine, and
   the first component is different from the second component.

Those in which the first component is alkylene diamine and the second component is an oxyalkylene diamine have proven to be particularly suitable copolymeric guanidine derivatives. Copolymeric guanidine derivatives in which the first component is hexamethylene diamine and the second component is triethylene glycol diamine in the mixture of amines are particularly preferable.

In the preparation of copolymers, the mixing ratio of the amines to be employed can be widely varied. However, those copolymeric guanidine derivatives are preferred in which the monomers of the mixture of amines, i.e., the first component and the second component, are in a molar ratio of from 4:1 to 1:4, preferably from 2:1 to 1:2.

The polymeric guanidine derivatives to be employed according to the invention preferably have an average molecular weight (weight average) within a range of from 500 to 7000, especially from 1000 to 5000, daltons.

In another preferred embodiment of the present invention, the polymeric guanidine derivative to be employed according to the invention is a mixture of at least 2 different polymeric guanidine derivatives. In a specific embodiment, the mixture of the polymeric guanidine derivatives comprises both a first homopolymer based on an alkylene diamine, preferably poly(hexamethylene guanidinium chloride), and a second homopolymer based on an oxyalkylene diamine, for example, poly[2-(2-ethoxy)ethoxyethyl)guanidinium chloride].

In a preferred embodiment, the polymeric guanidine derivative comprises the first homopolymer and the second homopolymer in a weight ratio of from 5:1 to 1:5, preferably from 1:1 to 1:4, especially from 1:2 to 1:4. In a particularly preferred embodiment, the mixture of polymeric guanidines comprises poly(hexamethylene guanidinium chloride) (first homopolymer) and poly[2-(2-ethoxy)ethoxyethyl)guanidinium chloride] (second homopolymer) in a weight ratio (of first homopolymer to second homopolymer) of from 1:1 to 1:5, preferably from 1:2 to 1:4, especially 1:3. Such mixtures exhibit an excellent property in terms of processability with plastic materials processed into medical articles.

Especially with respect to the biocidal activity and the leaching behavior in the incorporation of the polymeric guanidine derivatives into thermoplastic polymers that are subsequently processed into medical articles, the homopolymeric guanidine derivatives based on oxyalkylene diamines, especially poly[2-(2-ethoxy)ethoxyethyl)guanidinium chloride], have proven particularly suitable.

The polymeric guanidines used according to the invention all have an antibacterial activity that can be described by means of the so-called “minimum inhibition concentration”. This represents the lowest concentration of the bactericide that inhibits the growth of bacteria in a particular solution. For testing the biocidal activity of the polymeric guanidine derivatives to be employed according to the invention, these compounds are added to a bacterial nutrient medium, preferably tryptic soy broth, and diluted to different concentrations. These solutions of different concentrations are inoculated with a suspension of Escherichia coli and incubated at 37° C. for 24 hours.

The “minimum inhibition concentration” (MIC) is the lowest concentration of the polyguanidine to be tested in the solution that still inhibits the growth of the bacteria. In the corresponding inhibiting solution, turbidity from the growth of the bacteria cannot be observed. A minimum inhibition concentration of less than 50 μg/ml is particularly favorable. Preferably, the polymeric guanidine derivatives to be used according to the invention have a minimum inhibition concentration of less than 10 μg/ml. The lower this concentration, the more effectively can the corresponding polymeric guanidine derivative be employed as a biocide.

In a preferred embodiment, the polymeric guanidine derivatives to be employed according to the invention have a minimum inhibition concentration of 50 μg/ml or less, preferably 30 μg/ml or less, more preferably 10 μg/ml or less.

The polymeric guanidine derivatives to be employed according to the invention can be prepared relatively simply. The polycondensation can be effected by mixing one equivalent of a guanidine acid addition salt with one equivalent of the mixture of amines, followed by heating, preferably within a range of from 140 to 180° C., and stirring the melt at elevated temperatures, preferably within a range of from 140 to 180° C., until the evolution of gas is complete. The polycondensation is usually effected within a period of several hours, during which the melt is preferably stirred in a temperature range of 140 to 180° C. A preferred reaction time is from 1 to 15 hours, preferably from 5 to 10 hours.

According to the invention, the polymeric guanidine derivatives are employed as additives in compositions for medical articles. Depending on the biocidal effectiveness of the polymeric guanidine derivatives and the type and structure of the medical article, the compositions for the medical article may contain the polymeric guanidine derivative in an amount of at most 10.0% by weight, especially from 0.01 to 6% by weight, and especially in an amount of from 1.0 to 4.0% by weight, respectively based on the composition for the medical article, in a preferred embodiment.

A particular advantage of the polymeric guanidine derivatives to be employed according to the invention is their capability of being incorporated in plastic materials, especially thermoplastic polymers, which often form the essential component of compositions for medical articles. Surprisingly, it has been found that not only can the polymeric guanidine derivatives be incorporated in plastic materials, especially thermoplastic polymer compositions, without a problem, but in addition, the mechanical properties, such as the tensile strength or bending resistance, are not substantially affected thereby. In addition, it has surprisingly been found that the use of the polymeric guanidine derivatives in compositions comprising thermoplastic polymers for medical articles results in extremely smooth surfaces during the processing, and in addition shows no leaching effect, i.e., the biocidal polymeric guanidine derivatives are not leached out from the polymer blend by fluids, such as water or ethanol. Nevertheless, the compositions, especially compositions comprising thermoplastic polymers for medical articles, that have been treated with the polymeric guanidine derivatives exhibit an excellent antimicrobial effectiveness.

In a preferred embodiment, the composition for medical articles further includes thermoplastic polymers, especially those selected from polyurethane, polyolefin, polyvinyl chloride, polycarbonate, polystyrene, polyethersulfone, silicone and polyamide. More preferably, the compositions for medical articles include polymers selected from polyurethane or polyethylene or polypropylene.

In addition, it has surprisingly been found that the polymeric guanidine derivatives to be employed according to the invention may also be covalently bonded to a plastic matrix to which they are added as additives. The covalent bonding of the polymeric guanidine derivatives can be effected, for example, by reactive processing at elevated temperatures, for example, above 100° C., preferably above 140° C., in the presence of suitable thermoplastic polymers.

It has been found that compositions containing the polymeric guanidine derivatives covalently bonded to the plastic material have further improved properties in terms of the smoothness of the tubular medical articles and also a further reduced leaching behavior.

In addition, the polymeric guanidine derivatives covalently bonded to plastic materials, preferably thermoplastic polymers, surprisingly exhibit improved biocompatibility, which makes these products particularly suitable for medical articles that come into contact with body fluids, for example, catheters.

Therefore, in a preferred embodiment, the polymeric guanidine derivatives are covalently bonded to the plastic material.

Preferably, at least 50% by weight, more preferably at least 90% by weight, of the polymeric guanidine derivatives added as additives according to the invention is covalently bonded to the plastic material.

In addition, the compositions for medical articles may contain further usual additives. These include, in particular, fillers that are inert under physiological conditions. Barium sulfate is particularly suitable. For example, a suitable BaSO₄ can be purchased from the company Sachtleben Chemie GmbH under the trade name Blancfix®. The fillers are preferably contained in an amount of from 10 to 35% by weight, based on the total mixture, in the compositions for medical articles. Advantageously, the fillers have an average particle size of from 0.01 μm to 10 μm.

However, in a preferred embodiment of the present invention, the composition is substantially free of silicate fillers, because these could adversely affect the surface smoothness and the leaching effect.

According to the present invention, “substantially free” means that the silicate fillers may be present in an amount below 1% by weight, preferably below 0.5% by weight, more preferably below 0.01% by weight, and especially free from any silicate fillers, the weight percentages being based on the total weight of the composition for the preparation of the tubular medical article.

Tubular medical articles within the meaning of the present invention are those medical articles that can conduct fluids. In particular, the medical articles are selected from the group consisting of catheters, central venous catheters, peripheral venous catheters, breathing tubes, stents, couplings, ports, conduit systems, connectors, spikes, valves, three-way stopcocks, syringes, conduits, injection ports, wound drains, thoracic drains and probes.

Particularly preferred tubular medical articles include catheters, especially those prepared by the extrusion of compositions including polyurethane and/or polyethylene.

The present invention further relates to a process for preparing a tubular medical article, comprising the following steps:
a) combining and mixing a polymeric guanidine derivative having biocidal activity that is obtainable by the polycondensation of a guanidine acid addition salt with a mixture of amines containing at least one diamine selected from the group consisting of alkylene diamine and oxyalkylene diamine, with at least one plastic material, preferably a thermoplastic polymer;
b) subjecting the mixture obtained under a) to one or more shaping methods to form a tubular medical article.

Preferred polymeric guanidine derivatives and preferred plastic materials are those mentioned above.

The mixing in step a) is preferably effected by melt-kneading. The polymeric guanidine derivative may be added as an aqueous solution to the molten plastic material, followed by mixing in an extruder. Preferably, the melt-kneading is performed at temperatures above 100° C., more preferably above 150° C.

Preferably, the polymeric guanidine derivative is subjected to the shaping method in step b) as pellets or as a master batch. The production of pellets can be effected by processes familiar to those skilled in the art of plastics technology. Preferably, the master batch is in the form of pellets containing the polymeric guanidine derivative in a concentration higher than the final concentration desired for the medical article. Therefore, when a master batch is employed, it is further diluted to the desired final concentration by further adding plastic material.

The shaping method employed in step b) of the process according to the invention is preferably an extrusion method. The latter can be used to prepare, for example, tubular components of the catheter.

The shaping methods are preferably performed at temperatures above the melting point of the mixture prepared in step a), more preferably in a temperature range above 160° C., even more preferably in a range of 180 to 260° C.

As the result, a material with antimicrobial properties is obtained in which the additive (polymeric guanidine derivative) is physically admixed, or may optionally be chemically bonded to the respective plastic material.

The present invention further relates to a tubular medical article comprising a polymeric guanidine derivative having biocidal activity that is obtainable by the polycondensation of a guanidine acid addition salt with a mixture of amines containing at least one diamine selected from the group consisting of alkylene diamine and oxyalkylene diamine, and at least one plastic material, preferably a thermoplastic polymer.

Preferred polymeric guanidine derivatives and preferred plastic materials are those mentioned above.

The thermoplastic polymer to be employed in the medical article according to the invention and in the process according to the invention is preferably a thermoplastic polyurethane. Polyurethanes obtainable from a combination of 4,4′-diphenylmethane diisocyanate (MDI) and a polyester- or polyether-based polyol have proven particularly suitable. Advantageously, the polyol includes a polytetramethylene glycol ether. Other suitable thermoplastic polymers that are preferably used in compositions for the medical articles according to the invention are selected, for example, from polyurethane, polyolefin, polyvinyl chloride, polycarbonate, polystyrene, polyethersulfone, silicone and polyamide. More preferably, the compositions for medical articles include polyurethane or polyethylene or polypropylene or polyamide.

A particularly suitable polyamide is obtainable under the trade name of Pebax® (Arkema). It is a polyamide that includes polyether blocks.

Preferred medical articles are those mentioned above. Particularly preferred medical articles are catheters.

The present invention further relates to a biocidal material comprising a thermoplastic polymer covalently linked with at least one polymeric guanidine derivative that is obtainable by the polycondensation of a guanidine acid addition salt with a mixture of amines containing at least one diamine selected from the group consisting of alkylene diamine and oxyalkylene diamine.

Preferred embodiments of the polymeric guanidine derivative and of the thermoplastic polymer are described above. Thermoplastic polymers selected from the group of polyurethanes, polyesters, polyamides, polycarbonates, polyureas, polyesteramides and especially polycondensates are particularly suitable for the biocidal material according to the present invention.

In the preparation of the biocidal materials according to the invention, the bonding of the polymeric guanidine derivative to the thermoplastic polymer is preferably effected under the conditions of reactive processing. Thus, the polymeric guanidine derivative is mixed with the thermoplastic polymer, and the reaction conditions are selected in such a way that covalent bonding of the polymeric guanidine derivative to the thermoplastic polymer occurs. This can be done, for example, by selecting the thermoplastic polymer to contain remaining reactive groups, such as isocyanate groups.

However, the bonding of the polymeric guanidine derivative may also be effected under selected conditions within the scope of a transcondensation. Particularly suitable thermoplastic polymers include aliphatic polyurethanes and/or aromatic polyurethanes.

Surprisingly, it has been found that the biocidal material of the present invention is obtainable, in particular, if the thermoplastic polymer is melt-extruded at temperatures above 120° C., preferably above 160° C., and an aqueous solution, preferably a 20 to 50% by weight aqueous solution, of the polymeric guanidine derivative is added. Just under these reaction conditions, the covalent bonding of the polymeric guanidine derivatives to the thermoplastic polymer could be observed.

Polyesters, polylactides, polycaprolactones, polyamides, polyesteramides, polycarbonates, polyurethanes and polyureas are particularly suitable for covalent bonding, especially during a melt extrusion.

Polyethylene terephthalate, polybutylene terephthalate, polyamide PA6, PA66, PA610, PA11, PA12, aliphatic and aromatic polycarbonates as well as aliphatic and aromatic polyurethanes are particularly preferred.

The biocidal material according to the invention can be processed in an excellent way, exhibits excellent smoothness, in particular, and also exhibits an extremely low leaching effect. The biocidal materials according to the invention can be used for preparing medical articles or any kind of medical commodities.

Preferably, the biocidal materials of the present invention are used for the preparation of medical articles, especially selected from the group consisting of central venous catheters; peripheral venous catheters; breathing tubes, stents; products for application in regional anesthesia, especially catheters, couplings, filters; products for infusion therapy, especially containers, ports, conduit systems, filters; accessories, such as connectors, spikes, valves, three-way stopcocks, syringes, conduits, injection ports; products of formulation, especially transfer sets, mixing sets; urological products, especially catheters, urine measuring and collecting devices; wound drains; wound dressing; surgical suture materials; implantation auxiliaries as well as implants, especially plastic implants, for example, hernia meshes, non-wovens, knitwear/knitted fabrics, ports, port catheters, vascular prostheses; disinfectants; disposable surgical instruments; thoracic drains; probes; catheters; housings of medical devices, especially infusion pumps, dialysis devices and screens; artificial dentures; containers for liquids, especially contact lens containers.

Further, the biocidal materials of the present invention may also be used for the preparation of accessory parts of medical products, such as injection-molded parts and other molded parts. The use of the biocidal materials of the invention as additives in coatings for surgical suture material is of particular importance.

A further preferred medical article is a wound dressing.

In particular, the biocidal material can be used for preparing tubular medical articles, especially as defined above. A particularly preferred tubular medical article is a catheter.

Further features, details and advantages of the invention can be seen from the wording of the claims and from the following description of Examples.

### EXAMPLES

### Example 1

### Influence of the Addition of a Polyguanidine to be Employed According to the Invention on the Mechanical Properties of Thermoplastic Polyurethanes (TPUs)

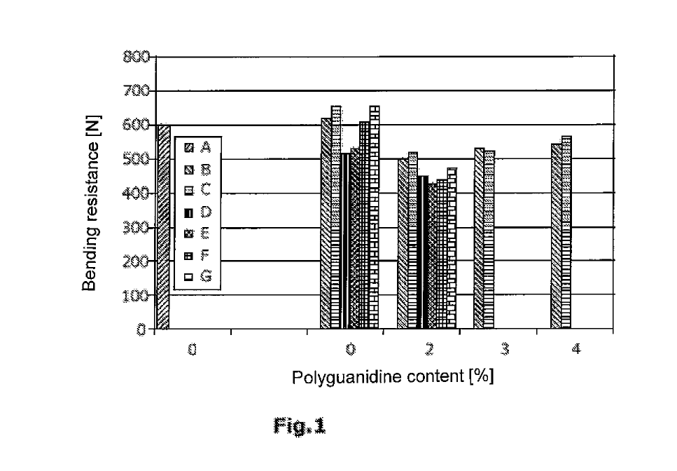

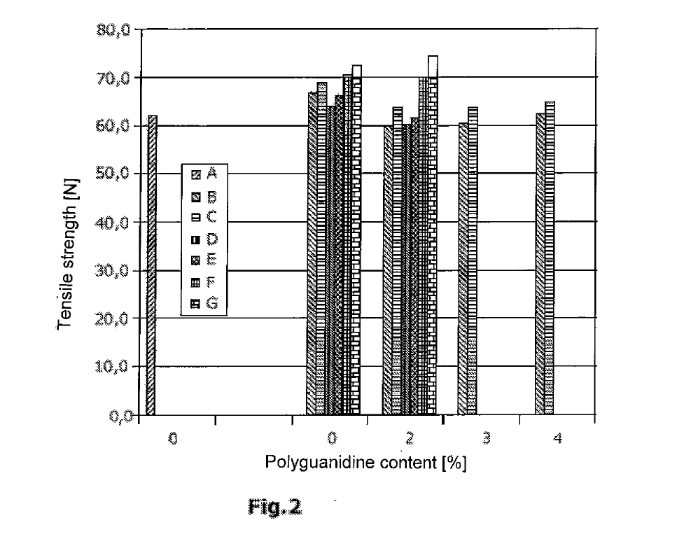

Specimens containing thermoplastic polyurethanes from different suppliers, each with 25% by weight barium sulfate and different amounts of the polymeric guanidine derivative poly[2-(2-ethoxy)ethoxyethyl)guanidinium chloride] (see FIGS. 1 and 2) are prepared. In order to additionally take the influence of the sterilization of plastic materials, which is common in medical technology, with ethylene oxide into account, one specimen each was sterilized with ethylene oxide, and a corresponding comparative specimen was not sterilized. The sterilization with ethylene oxide proceeds by a process known to the skilled person by gassing the specimens with ethylene oxide at 40 to 50° C.

TABLE 1
Different TPUs with BaSO₄

| | | | Sterilization with |
|---|---|---|---|
| Specimen | BaSO₄¹ | TPU | ethylene oxide |
| A | 25% by weight | Pellethane ®² | — |
| B | 25% by weight | Pellethane ® | — |
| C | 25% by weight | Pellethane ® | yes |
| D | 25% by weight | Elastollan ®³ | — |
| E | 25% by weight | Elastollan ® | yes |
| F | 25% by weight | Tecothane ®⁴ | — |
| G | 25% by weight | Tecothane | yes |

¹Barium sulfate with an average grain size of 0.7 μm
²Pellethane ® (thermoplastic polyurethane from Dow Chemicals, U.S.A.)
³Elastollan ® (thermoplastic polyurethane from Elastogran GmbH, Germany)
⁴Tecothane ® (thermoplastic polyurethane from Thermetics Polymer Products, U.S.A.)

The results of the bending resistance measurement and tensile strength are shown in FIGS. 1 and 2, respectively.

It is found that the addition of the employed polyguanidine as an additive to plastic materials has no significant influence on the mechanical properties of the plastic materials.

### Example 2

### Production of a Triple-Lumen Catheter Tube

The thermoplastic polyurethane Pellethane® 2363-90A (Lubrizol Advanced Materials; U.S.A.) is mixed with 25% by weight barium sulfate having an average grain size of 0.7 μm and 2% by weight poly[2-(2-ethoxy)ethoxyethyl)guanidinium chloride], and the mixture was extruded. The extrusion was performed using the extruder Maillefer type ED45-30D.

### Example 3

### Comparative Example

A piece of catheter tube according to Example 2 is prepared, but without adding the polyguanidine.

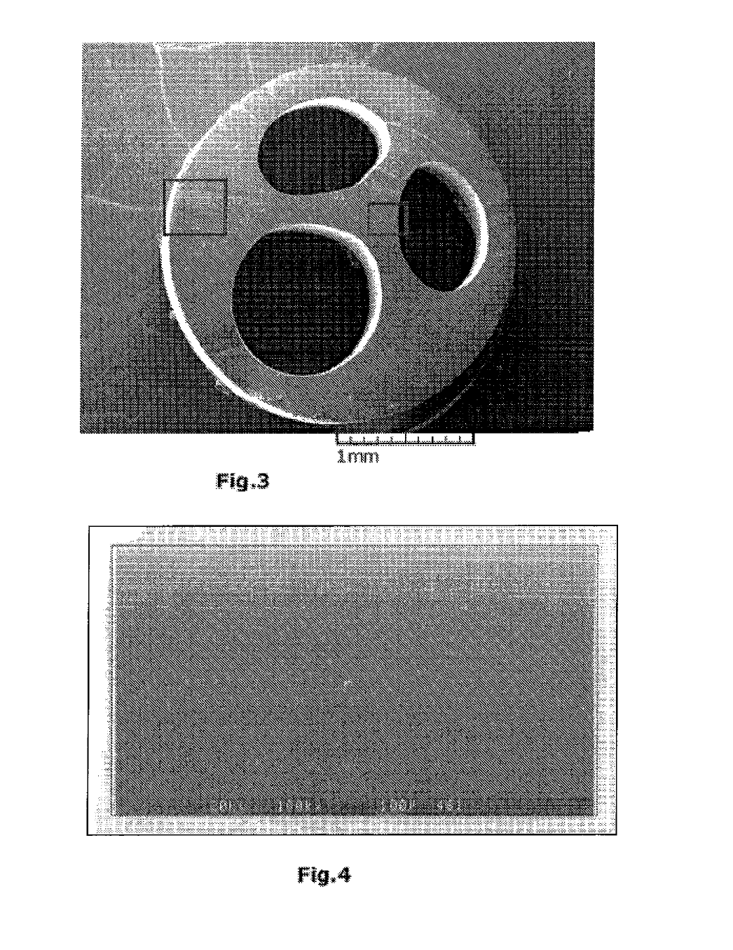

FIG. 3 shows the piece of triple-lumen catheter tube prepared according to Example 2. A homogeneous distribution of the polyguanidine employed in the extruded polymer material could be observed.

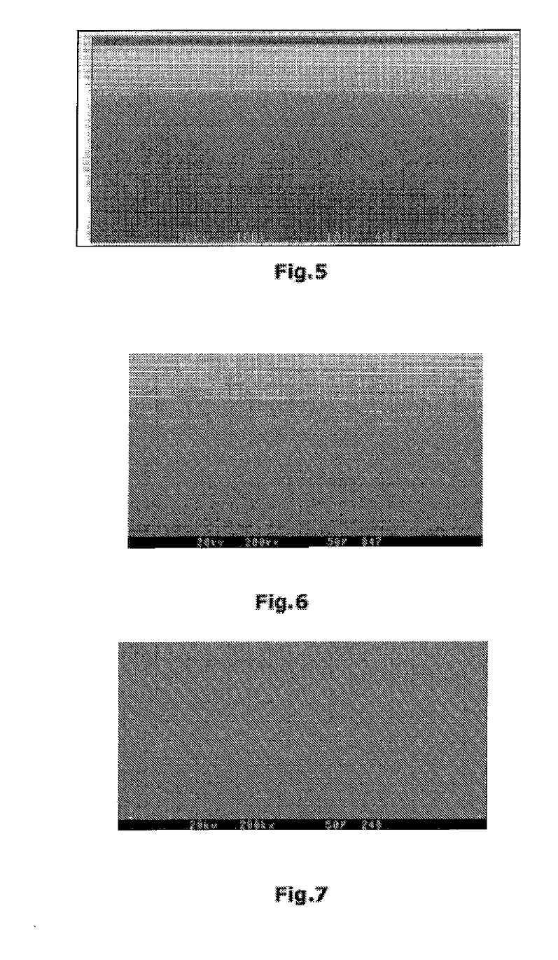

The pieces of catheter tube prepared according to Examples 2 and 3 were examined by scanning electron micrographs. FIGS. 4 and 6 show scanning electron micrographs with different definitions of pieces of catheter tube prepared according to Comparative Example 3. FIGS. 5 and 7 show surface micrographs of the piece of catheter tube prepared according to Example 2.

It is found that the addition of polyguanidine results in a very smooth plastic surface, which is important, in particular, to the use of catheters in order to reduce or avoid platelet aggregation and biofilm formation.

### Example 4

### Preparation of a Central Nervous Catheter

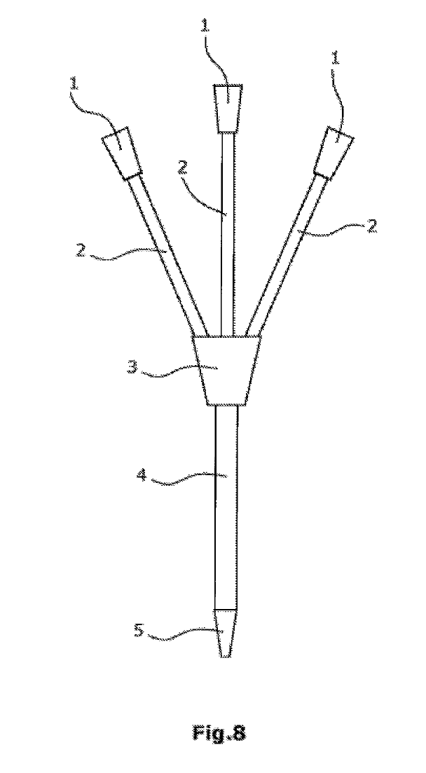

FIG. 8 shows a central nervous catheter with a triple-lumen catheter tube (4), port and supply lines (2), the channel branch (3), connecting pieces (1) and a tip (5). Parts (1) to (5) may respectively consist of different plastic materials, and these in turn may respectively comprise the polyguanidines to be used according to the invention as additives.

Prefabricated plastic pellets that already contain the polymeric guanidine derivative to be employed according to the invention in the desired concentration is employed as the starting material for producing the catheter.

The tubular components of the catheter (catheter tube (4), supply line (2) and tip (5)) are produced as semifinished products in extrusion. The plants used for extruding the compounds (pellet production) do not differ from the plants for processing the respective base materials. Thus, the tube segments can be prepared by a process known to those skilled in the art of plastics technology.

In detail, the central venous catheter shown in FIG. 8 is prepared as follows:

### a) Catheter Tube (4):

Cutting the tube to the desired length, slitting the distal end, punching the lateral openings of the proximal end. The plants and processes employed for the processing do not differ from those used for processing tube pieces without antimicrobial treatment. All the plants and processes are state of the art.

### b) Supply Line (2):

Cutting the tube to the desired lengths. The plants and processes employed for the processing do not differ from those used for processing tube pieces without antimicrobial treatment. All the plants and processes are state of the art.

### c) Soft Tip (5):

Cutting the tube to the desired lengths. The plants and processes employed for the processing do not differ from those used for processing tube pieces without antimicrobial treatment. All the plants and processes are state of the art.

These process steps result in tube segments whose geometry corresponds to the geometry required for the final product.

### d) Injection-Molding the Ports Around the Feeding Lines:

The ports are produced by injection molding; this process step integrates the assembly of the ports with the feeding lines. The plants and processes employed for the processing do not differ from those used for processing tube pieces and pellets without antimicrobial treatment. All the plants and processes are state of the art.
This process step results in feeding lines that are permanently assembled with the ports.

### e) Forming the Tip:

The soft tip is given its final shape by thermoforming. This process step integrates the assembly of the soft tip and the catheter tube. The joining process corresponds to the welding of plastic materials. The plants and processes employed for the processing do not differ from those used for processing tube pieces without antimicrobial treatment. All the plants and processes are state of the art.
This process step results in a soft tip with the required shape permanently connected with the catheter tube.

### f) Injection-Molding the Channel Branch Around the Catheter Tube and the Feeding Lines:

The channel branch is produced by an injection molding process. This process step integrates the assembly of the catheter tube and the channel branch as well as the feeding lines and channel branch. The plants and processes employed for the processing do not differ from those used for processing tube pieces and pellets without antimicrobial treatment. All the plants and processes are state of the art.

This process step results in a catheter corresponding to a central venous catheter in terms of dimensions and shape.

### Example 5

The following tubular components of a central venous catheter are prepared according to Example 4:
C1: a port line of Pellethane® without polyguanidine additive
C2: catheter tube (4) of Pellethane® 2363-90A with 25% by weight barium sulfate (grain size: 0.7 μm) and 2% by weight poly[2-(2-ethoxy)ethoxy-ethyl)guanidinium chloride].
C3: like C2, except that 3% by weight poly[2-(2-ethoxy)ethoxyethyl)-guanidinium chloride] was added
C4: like C2, except that 4% by weight poly[2-(2-ethoxy)ethoxyethyl)-guanidinium chloride] was added
C5: like C2, except that Elastolan® was employed instead of Pellethane®
C6: soft tip of Tecothane®, 25% by weight barium sulfate (grain size: 0.7 μm) and 2% by weight poly[2-(2-ethoxy)ethoxyethyl)guanidinium chloride].

The tubular components C1 to C6 were tested for their antimicrobial effectiveness within the scope of a proliferation test (Bechert et al. “A new method for screening anti-effective biomaterials”; Nature Medicine 2000, 6(9): 1053-1056) on various bacterial strains. The samples are said to be “antimicrobial” if they show a reduction of the ability to reproduce of the respective germs by at least three log steps.

Table 2 shows the result.

TABLE 2
Antimicrobial effectiveness of medical articles against different bacterial strains

| | | | MRSA (methicillin- | | |
|---|---|---|---|---|---|
| | | | resistant | Candida | |
| Catheter | Staphylococcus | Staphylococcus | Staphylococcus | albicans | |
| component | epidermidis | aureus | aureus) | (fungus) | E. coli |
| C1 | not | not | not | not | not |
| | antimicrobial | antimicrobial | antimicrobial | antimicrobial | antimicrobial |
| C2 | antimicrobial | antimicrobial | antimicrobial | antimicrobial | not |
| | | | | | antimicrobial |
| C3 | antimicrobial | antimicrobial | antimicrobial | antimicrobial | antimicrobial |
| C4 | antimicrobial | antimicrobial | antimicrobial | antimicrobial | antimicrobial |
| C5 | antimicrobial | antimicrobial | antimicrobial | antimicrobial | antimicrobial |
| C6 | antimicrobial | antimicrobial | antimicrobial | antimicrobial | antimicrobial |

As can be seen from Table 2, the tubular catheter components treated with the polyguanidines to be used according to the invention are highly effective against the strains Staphylococcus epidermidis, Staphylococcus aureus, MRSA (methicillin-resistant Staphylococcus aureus), E. coli, and Candida albicans.

Samples C2 and C4 were additionally subjected to a hemolysis test and a leaching test with ethanol and water. The leaching test was performed by extracting the test material with distilled water containing 5% ethanol over a period of 24 hours at a temperature of 37° C. The surface to volume ratio of the test materials was 3 cm²/ml. The extract is subsequently analyzed for polyguanidines. Thus, 0.2 g of potassium hexacyanoferrate and 0.2 g of sodium nitroprusside are added to 4 ml of a 6 N sodium hydroxide solution, and the solution thus obtained is filled with deionized water to 100 ml. One milliliter of the extract is dried and subsequently dissolved in 1 ml of deionized water. Subsequently, 4 ml of the previously prepared detection reagent is added, followed by analyzing the solution by means of UV/Vis spectroscopy.

The hemolysis test showed that no substances were released in hemolytic concentrations. In addition, the leaching test showed that the extraction with ethanol/water did not result in a detection of the polyguanidine employed.

### Example 6

For the Introcan® Safety catheter from the company B. Braun Melsungen (Germany), both the catheter and the housing were treated with the polyguanidine to be employed according to the invention.

Thus, the following medical articles were prepared:
D1: Tecoflex® catheter made of polyurethane both with and without silicone oil coating, and without polyguanidine additive
D2: Tecoflex® catheter made of polyurethane with 2% by weight poly[2-(2-ethoxy)ethoxyethyl)guanidinium chloride] and both with and without silicone oil coating
D3: Tecoflex® catheter made of polyurethane with 4% by weight poly[2-(2-ethoxy)ethoxyethyl)guanidinium chloride] and both with and without silicone oil coating
D4: Introcan® Safety housing made of polypropylene without polyguanidine additive
D5: Introcan® Safety polypropylene housing with 2% by weight poly[2-(2-ethoxy)ethoxyethyl)guanidinium chloride]
D6: Introcan® Safety polypropylene housing with 4% by weight poly[2-(2-ethoxy)ethoxyethyl)guanidinium chloride]

The antimicrobial effectiveness of the medical articles was summarized in Table 3 by using a proliferation test (Bechert et al. “A new method for screening anti-effective biomaterials”; Nature Medicine 2000, 6(9): 1053-1056). In addition, Example D2 did not show a leaching effect (test description, see Example 2).

TABLE 3
Antimicrobial effectiveness of medical articles
against different bacterial strains

| | | | Candida albicans |
|---|---|---|---|
| Sample | Staph. epidermidis | E. coli | (fungus) |
| D1 | not antimicrobial | not antimicrobial | not antimicrobial |
| D2 | antimicrobial | antimicrobial | antimicrobial |
| D3 | antimicrobial | antimicrobial | antimicrobial |
| D4 | not antimicrobial | not antimicrobial | not antimicrobial |
| D5 | antimicrobial | antimicrobial | antimicrobial |
| D6 | antimicrobial | antimicrobial | antimicrobial |

1. Use of at least one polymeric guanidine derivative having biocidal activity that is obtainable by the polycondensation of a guanidine acid addition salt with a mixture of amines containing at least one diamine selected from the group consisting of alkylene diamine and oxyalkylene diamine, as an additive in a composition for tubular medical articles.

2. The use according to claim 1, characterized in that said guanidine acid addition salt is guanidine hydrochloride.

3. The use according to claim 1, characterized in that said alkylene diamine is a compound of general formula
NH₂(CH₂)ₙNH₂
in which n represents an integer of from 2 to 10.

4. The use according to claim 1, characterized in that said oxyalkylene diamine is a compound of general formula
NH₂[(CH₂)₂O)]ₙ(CH₂)₂NH₂
in which n represents an integer from 2 to 5.

5. The use according to claim 1, characterized in that said polymeric guanidine derivative is a homopolymer.

6. The use according to claim 1, characterized in that said mixture of amines contains a first component and at least one second component, wherein
the first component is a diamine selected from the group consisting of alkylene diamine and oxyalkylene diamine, and wherein
the second component is a diamine selected from the group consisting of alkylene diamine and oxyalkylene diamine, and
the first component is different from the second component.

7. The use according to claim 6, characterized in that said first component is alkylene diamine and said second component is an oxyalkylene diamine.

8. The use according to claim 7, characterized in that said alkylene diamine is hexamethylene diamine and said oxyalkylene diamine is triethylene glycol diamine.

9. The use according to claim 6, characterized in that said first component and said second component are present in a molar ratio of from 4:1 to 1:4.

10. The use according to claim 1, characterized in that said polymeric guanidine derivative has an average molecular weight within a range of from 500 to 7000.

11. The use according to claim 1, characterized in that said polymeric guanidine derivative is present in an amount of at most 10.0% by weight, respectively based on the composition for the medical article.

12. The use according to claim 1, characterized in that said composition further includes plastic materials, especially thermoplastic polymers, preferably selected from polyurethane, polyolefin, polyvinyl chloride, polycarbonate, polystyrene, polyethersulfone, silicone and polyamide.

13. The use according to claim 12, characterized in that said polymeric guanidine derivative is covalently bonded to said plastic material.

14. The use according to claim 1, characterized in that said composition is substantially free of silicate fillers.

15. The use according to claim 1, characterized in that said medical article is selected, in particular, from the group consisting of catheters, central venous catheters, peripheral venous catheters, breathing tubes, stents, couplings, ports, conduit systems, connectors, spikes, valves, three-way stopcocks, syringes, conduits, injection ports, wound drains, thoracic drains and probes.

16. A process for preparing a tubular medical article, comprising the following steps:
a) combining and mixing a polymeric guanidine derivative having biocidal activity that is obtainable by the polycondensation of a guanidine acid addition salt with a mixture of amines containing at least one diamine selected from the group consisting of alkylene diamine and oxyalkylene diamine, with at least one plastic material, preferably a thermoplastic polymer;
b) subjecting the mixture obtained under a) to one or more shaping methods to form a tubular medical article.

17. The process according to claim 16, characterized in that said mixing in step a) is effected by melt-kneading, preferably at temperatures above 100° C.

18. A tubular medical article comprising a polymeric guanidine derivative having biocidal activity that is obtainable by the polycondensation of a guanidine acid addition salt with a mixture of amines containing at least one diamine selected from the group consisting of alkylene diamine and oxyalkylene diamine, and at least one plastic material, preferably a thermoplastic polymer.

19. The tubular medical article of claim 18, characterized in that the polymeric guanidine derivative is covalently bonded to the plastic material.

20. The tubular medical article of claim 18, characterized by being a catheter.
